(19) United States
(12) Patent Application Publication
(10) Pub. No.: US 2015/0133768 A1
(43) Pub. Date: May 14, 2015

(54) USE OF NON-METALLIC CEST AGENTS FOR MRI MONITORING OF NANOPARTICLE DELIVERY

(71) Applicants: THE JOHNS HOPKINS UNIVERSITY, Baltimore, MD (US); KENNEDY KRIEGER INSTITUTE, INC., Baltimore, MD (US)

(72) Inventors: Michael T. McMahon, Columbia, MD (US); Kannie W.Y. Chan, Baltimore, MD (US); Guanshu Liu, Nottingham, MD (US); Jeff W.M. Bulte, Fulton, MD (US); Nikita Oskolkov, Reisterstown, MD (US)

(21) Appl. No.: 14/395,127

(22) PCT Filed: Apr. 17, 2013
(86) PCT No.: PCT/US2013/036904

§ 371 Date: Oct. 17, 2014

Related U.S. Application Data
(60) Provisional application No. 61/625,426, filed on Apr. 17, 2012.
(60) Provisional application No. 61/655,215, filed on Jun. 4, 2012.

(51) Int. Cl. A61K 49/18; A61K 31/704; A61K 31/337; A61B 5/055

(52) U.S. Cl. CPC A61K 49/1818; A61B 5/055; A61K 31/704; A61K 31/337
USPC 600/420; 514/34; 514/449; 424/450

(57) ABSTRACT

The present invention includes drug-loaded, polymer nanoparticles and liposomes further incorporating a non-paramagnetic, bioorganic CEST agent. The CEST agent allows for an alternative approach to accomplish MR-compatible in vivo tracking of drug-loaded polymer nanoparticles and liposomes, including simultaneous multi-color mapping of more than one particle type, or of the same particle type delivered via two different routes (e.g., systemic versus local). Additionally, the present invention can include a library of biodegradable diamagnetic (DIA)CEST agents. These DIACEST agents can be incorporated into nanoparticle-based delivery systems, such as stealth liposomes loaded with doxorubicin and stealth polymer nanoparticles loaded with paclitaxel. These systems can be tracked, according to an embodiment of the present invention using CEST-based MRI (compared to SPECT/CT) as a method to monitor the efficiency with which the nanoparticles reach the targeted tumors and how long they persist. Measured particle persistence times are also used to guide the spacing between doses.

### CROSS REFERENCE TO RELATED APPLICATIONS

This application claims the benefit of U.S. Provisional Patent Application No. 61/625,426 filed on Apr. 17, 2012, and U.S. Provisional Patent Application No. 61/655,215 filed on Jun. 4, 2012 both of which are incorporated by reference, herein, in their entirety.

### GOVERNMENT SUPPORT

This invention was made with government support under R01 EB015031 and 1R01EB012590 both awarded by the National Institutes of Health. The government has certain rights in the invention.

### FIELD OF THE INVENTION

The present invention relates generally to medical imaging. More particularly the present invention relates to agents for and methods of monitoring nanoparticle delivery.

### BACKGROUND OF THE INVENTION

A significant challenge in cancer therapy is the targeting of a high dose of active agent (e.g., chemotherapeutics and cytokines) to tumors and any metastases, while minimizing exposure to healthy tissues. Liposomes are nanoscale biodegradable particles composed of lipids. Long-circulating “stealth” liposomes containing doxorubicin (Doxil®) are now approved for treatment of ovarian cancer, AIDS-related Kaposi's sarcoma and multiple myeloma. Liposomes are used to improve the delivery of mostly water-soluble drugs. Nanosized drug delivery particles possess an innate ability to target tumors via leaky tumor vasculature. Once at the tumor, they release their drug payload in a steady fashion over a prolonged period of time, which reduces the risk of adverse reactions and greatly improves drug efficacy. Originally, liposomes were composed of just a lipid coat and they were not able to avoid rapid elimination by the reticulo-endothelial system (RES). Addition of small amounts of polyethylene glycol (PEG) was found to be highly advantageous in allowing them sufficient time to circulate so that they can enter tumors via the leaky vasculature. There have been multiple successful medical products, with doxorubicin the most commonly used liposome encapsulated anti-tumor drug.

By way of example, cervical cancer is among the leading causes of death from cancer in women worldwide. The majority of early stage tumors are treated with surgery, and radiation therapy is reserved for localized relapse, while those presenting with advanced cancers are given concurrent chemoradiation as the standard of care. Even though treatment for cervical cancer has improved considerably, challenges still remain for the management of both early stage and advanced disease. For example, infertility remains a principal concern for women with cervical dysplasia who receive surgical treatment. In certain cases fertility-preserving treatment is possible, but risks, including miscarriage, intrauterine growth retardation, and preterm delivery during pregnancy still exist. Many patients with advanced cervical cancer fail to respond to recommended therapy, resulting in disease progression and ultimately death. Conventional chemotherapy (i.e., without drug delivery systems) suffers from several limitations, including adverse side effects and low drug concentration in the tumor.

For patients with advanced disease, or with early-stage disease but high risk of postoperative recurrence, systemic chemotherapy is standard treatment. General features of tumors include leaky blood vessels and poor lymphatic drainage. This increased permeability of blood vessels in tumors is characteristic of rapid and defective angiogenesis. Nanoparticles of up to several hundred nanometers in diameter can extravasate into tumor tissues via leaky vessels via the “EPR effect” (enhanced permeability and retention effect), where the dysfunctional lymphatic drainage of tumors retains the particles. Accumulated nanoparticles then release drugs into the vicinity of the tumor cells. Nanoparticles ˜200 nm in size can preferentially accumulate and be retained in TC1 murine cervical tumors following systemic or local administration. Although passive targeting approaches form the basis of clinical therapy, they suffer from several limitations such as insufficient EPR effect exhibited by certain tumors or heterogeneity in vessel permeability throughout a tumor. For example, in the case of Doxil treatment, there is variability in the tumor leakiness between patients, which allows only a subset of patients to benefit from treatment. Sorting out which patients would be good candidates for treatment, and confirming that drug-loaded particles arrived and were retained at the tumor site, would be significant advancements.

Early stage cervical cancer is largely insensitive to systemic chemotherapy since the small tumors often have limited blood supply (insignificant angiogenesis). As a result, the majority of cases are treated with surgery and/or radiation therapy. Thus, methods that could provide localized chemotherapy in the CV tract, which may reduce the adverse side effects common with systemic chemo and/or improve the efficacy due to greater drug concentration at the tumor, are needed. Sustained levels of drug in the vicinity of tumors is well established to improve chemotherapy by killing cancer cells that enter and exit the sensitive phase of the cell cycle in an asynchronous manner. However, the primary challenge with local administration of free chemo drugs to the female reproductive tract is the short duration with which an adequate concentration of the drugs can be maintained. In the female reproductive tract, nanoparticles are small enough to penetrate the mucus barrier (if they do not adhere to it), but too large to permeate the underlying cervicovaginal epithelium. This “selective permeability”, coupled with a tailored drug release profile, may allow mucus penetrating particle (MPP) based systems to provide an effective drug concentration over a prolonged period of time in the female reproductive system. Indeed, much research has been performed to evaluate the potential of conventional nanoparticles (e.g. those made of PLGA and other non-penetrating polymers) to provide sustained drug release in the vaginas of animals. However, conventional nanoparticles (CPs) are easily immobilized by mucus, leading to their rapid elimination from the CV tract by natural mucus clearance mechanisms. This strongly limits their use in the treatment of various mucosal diseases, including cervical cancer. In contrast, by penetrating the mucus layer in the vagina, mucus penetrating particles (MPP) are retained for a much longer duration.

Nanotechnology has the potential to revolutionize cancer diagnosis and therapy. However, nanoparticles encounter numerous barriers en route to the diseased tissue, such as mucosal barriers (reducing effectiveness of locally-administered nanoparticle therapies) and non-specific uptake by immune cells, primarily in the liver (reducing effectiveness of systemically-administered nanoparticle therapies), which may lead to unpredictable outcome of treatment. Nanoparticle systems that resist mucosal and immune cell clearance have been developed. However, to improve the effectiveness of these treatment modalities, it is important to develop improved molecular imaging technology to allow monitoring of the interactions of the nanoparticles with barriers in the body, the efficiency with which these particles reach their target and the length of time they remain at the target (e.g., cervical tumor). Such monitoring is available to an extent. However, in its current form it carries risks for the patient.

Biodegradable stealth polymeric particles complement liposomes in that they typically can be used to more efficiently encapsulate hydrophobic drugs, like paclitaxel, while providing excellent storage stability and a more controlled release of drug. Polymer nanoparticles can be composed of a wide range of biocompatible polymers, including poly(lactide-co-glycolide) (PLGA), a polymer that has been used safely in humans for years in products ranging from sutures to particle forms, such as the Lupron Depot used for prostate cancer.

However, when doctors administer drug-loaded nanoparticles, such as Doxil® liposomes, to a patient, there is currently no way to confirm that the particles were administered properly, that they reached their target (tumor), or how long they persist in the tumor (which could guide dosing regimens). Incorporation of MRI contrast agents that can provide this information built into nanoparticles is expected to greatly improve therapy.

In order to obtain information about targeted delivery of drugs by nanoparticles, it is important to have direct (preferably real-time) feedback on: (i) whether or not the particles were successfully administered, (ii) whether or not the particles arrived at the tumor, and (iii) how long the particles lasted at the tumor site (persistence). As such, it is important to have in vivo imaging technology with the spatial resolution to localize these particles and sufficient contrast to detect them despite their low concentrations. Such technology should preferably be non-invasive, give high soft tissue contrast, and have the ability to be repeated over a prolonged treatment timescale (a month or perhaps longer). MRI possesses many of these characteristics and has been used to define lymph nodes status or to assess the extent of local disease in cervical cancer. Currently, two major classes of MR contrast agents are in routine use: paramagnetic agents (e.g. chelates of Gd or Mn, or Mn particles), producing large positive signal enhancement from decreasing T₁, and superparamagnetic agents, such as iron oxide particles₁₆₁₇, which produce large negative T₂ contrast. Using these, MRI has been able to monitor liposome location by loading MnCl₂, Mn-DTPA, Gd-DTPA, Gd-HP-DO3A, or even Mn bound to proteins in the particle interior. These agents have sufficient sensitivity to highlight tumors in liver and brain. Covalently attaching paramagnetic molecules to the liposome lipid bilayer results in higher relaxivity than when sequestering them in the interior. This approach has been applied to detect lymph nodes₃₅ and highlight tumors₃₄. In recent MRI studies of liposomes, changes in relaxivity due to contrast agent being released from the interior of liposomes to the exterior was utilized. One study co-loaded Mn with doxorubicin, and were able to monitor release of Mn (as a possible surrogate for doxorubicin release) based on the T₁ shortening that resulted.

While Magnetic Resonance Imaging (MRI) is readily available as a clinical tool, its contrast agents contain paramagnetic metals that need to be administered in relatively high doses (compared to PET/SPECT and optical methods). The safety of these metals has recently been questioned. MR contrast agents that contain Gd may be toxic to the kidneys, raising concerns since these agents typically are administered in relatively high doses or when they stay around longer, raising the risk of metal release. MR agents based on paramagnetic metals also have the significant limitation that they provide only one type of contrast (signal intensity change). If it were instead possible to develop bioorganic biodegradable compounds tailored for multi-color MRI detection, it may be possible to gather information regarding delivery efficiency and persistence in a safe manner that allows simultaneous tracking of more than one drug/nanoparticle.

Another type of MRI contrast has been developed called Chemical Exchange Saturation Transfer (CEST). CEST agents have exchangeable protons with different characteristic MR frequencies (multiple “colors”) that can be used to selectively highlight different tissues and agents (e.g., tumor and nanoparticles) simultaneously.

Recently, more classes of MR agents have become available, such as based on magnetic isotope labeling (e.g. F “hot spot” imaging using particles loaded with perfluorocarbons), and so-called chemical exchange saturation transfer (CEST) contrast agents. CEST agents are especially powerful in that they can be selectively labeled using frequency-specific radio-frequency (rf) saturation of exchangeable protons on the agents. Chemical exchange causes these protons to transfer this saturation to water protons. Because the water proton pool is very large, unlabeled water protons move back to the agent and the process repeats itself, leading to large sensitivity enhancements, ultimately allowing MRI detection. Paramagnetic agents with appropriately shifted exchangeable groups, termed “PARACEST” agents can also be used in MR. CEST can detect micromolar concentrations of polypeptide gene carriers and close to nanomolar concentrations of polynucleotides. These non-metallic diamagnetic compounds are called DIACEST agents

It would therefore be advantageous to provide drug delivery with incorporated CEST agents for use related methods of monitoring nanoparticle delivery.

### SUMMARY

According to a first aspect of the present invention an agent for use in conjunction with magnetic resonance (MR) imaging includes a biocompatible chemical exchange saturation transfer (CEST) agent. The biocompatible CEST agent is configured to create contrast in an MR image detectable using a saturation transfer CEST method of MR imaging. The agent also includes a drug delivery system and a therapeutic agent. The biodegradable CEST agent, the drug delivery system, and therapeutic agent are combined to form a particle.

In accordance with an aspect of the present invention, the particle further takes the form of a nanoparticle. The CEST agent can take the form of a non-metallic DIACEST agent. The agent is configured for use as systemic nanoparticle-based chemotherapy, and alternately, the agent is configured for use as local nanoparticle-based chemotherapy. The CEST agent further takes the form of at least one of a polypeptide or organic heterocycle. Additionally, the CEST can take the form of at least one of a peptide that is ring NH-rich, backbone NH-rich, guanidyl NH₂-rich, or OH-rich. The CEST agent further includes a macromolecule with multiple amide or imino groups. The drug delivery system comprises a mucus penetrating particle.

In accordance with another aspect of the present invention, the drug delivery system takes the form of one of a stealth liposome or a stealth poly(lactic-co-glycolic acid)-co-polyethylene glycol (PLGA-PEG) particle. The thereapeutic agent takes the form of one of doxorubicin or paclitaxel.

In accordance with still another aspect of the present invention, a method for tracking a delivery of a therapeutic agent in a subject includes providing a particle containing a biocompatible CEST contrast agent, a therapeutic agent, and a drug delivery system. The biocompatible CEST agent is configured to create contrast in an MR image detectable using a saturation transfer CEST method of MR imaging. The particle is delivered to the subject and saturation transfer CEST method of MR imaging is used to obtain an MR image of the subject. The images of the subject are used to track the delivery of the therapeutic agent using the contrast created by the CEST agent.

In accordance with yet another aspect of the present invention, the method further includes using the particle to treat cancer, and more particularly, can be used to treat cervical cancer. The method can also include delivering the particle to the subject systemically, or alternately, can include delivering the particle to the subject locally. A library of available CEST contrast agents can be built for use in conjunction with the method. The method can also include using multicolor MR imaging. In addition, information from the image of the subject can be used to determine dose frequency for the therapeutic agent and also clearance of the therapeutic agent.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1 illustrates the approach for detecting backbone amide NH protons in a peptide.

FIGS. 2A-2C, illustrate a list of 30 of such CEST peptides, and compares the sensitivity for three varieties: NH-rich (FIG. 2A), gNH2-rich (FIG. 2B), and OH-rich peptides (FIG. 2C).

FIGS. 3A-3D illustrate four pyrimidine and imidazole compounds tested to determine the best substitutions.

FIGS. 4A and 4B illustrates the CEST color spectrum for this range of exchangeable protons in vitro prior to incorporation into nanocarriers, demonstrating that the CEST contrast curves (MTRasym curves illustrated in FIG. 1) are quite different for these protons.

FIG. 5 illustrates that paclitaxel-loaded MPP inhibits tumor growth in an orthotopic murine cervical cancer model, while other local treatments, including paclitaxel-loaded CP and free Taxol®, are much less effective.

FIG. 6 illustrates multicolor MR imaging using a phantom containing three different peptide-based CEST agents.

FIG. 7 illustrates results for three such DIACEST liposomes prepared containing L-arginine (Larg), poly-L-lysine (PLL), and glycogen (Glyc), using starting solutions containing 100 mg/ml Larg, 25 mg/mL PLL (MW=15 kD) or 100 mg/ml Glyc (MW=25 k-100 k), respectively.

FIG. 8 illustrates SPECT data that shows most of the liposomes remain near the injection site during the time period of the study, with the popliteal lymph node showing the highest uptake outside the foot.

FIG. 9 illustrates histology was performed to determine how the liposomes were distributed within these nodes.

FIGS. 10A-10C illustrate an example of the types of images acquired for the PLL liposomes, which are clearly detected in the popliteal lymph node on the inject side.

FIG. 10D illustrates the CEST contrast on the injection and control side for all three CEST formulations.

FIG. 11 illustrates images showing growth of tumor cells monitored every two days by live animal bioluminescence imaging.

FIG. 12 illustrates images of tumor free and tumor bearing mice.

FIG. 13 illustrates images of DIACEST PLGA-PEG particles administered locally.

FIG. 14 illustrates “multicolor” CEST imaging to discriminate between PLL and Larg liposomes.

### DETAILED DESCRIPTION OF THE PREFERRED EMBODIMENTS

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention includes drug-loaded, polymer nanoparticles and liposomes further incorporating a non-paramagnetic, bioorganic CEST agent. The CEST agent incorporated into the drug delivery system allows for an alternative approach to accomplish MR-compatible in vivo tracking of drug-loaded polymer nanoparticles and liposomes, including simultaneous multi-color mapping of more than one particle type, or of the same particle type delivered via two different routes (e.g., systemic versus local). Additionally, the present invention can include a library of biodegradable diamagnetic (DIA)CEST agents. These DIACEST agents can be incorporated into nanoparticle-based delivery systems, such as stealth liposomes loaded with doxorubicin and stealth polymer nanoparticles loaded with paclitaxel. These systems, generically referred to as “particles” throughout this application can be tracked, according to an embodiment of the present invention using CEST-based MRI (compared to SPECT/CT) as a method to monitor the efficiency with which the nanoparticles reach the targeted tumors and how long they persist. Measured particle persistence times can also be used to guide the spacing between doses.

Generally, a particle, according to an embodiment of the present invention will include a CEST contrast agent, a disease treatment agent, and a liposome or polymeric drug delivery system. While many examples throughout this application utilize cervical cancer as the disease for treatment, it should be noted that, the particles of the present invention, as well as the method for tracking these particles are not limited to just the treatment of and use with cervical cancer. These particles could be used in association with any ailment or disease known to one of skill in the art.

One aspect of the present invention includes libraries of polypeptides and organic heterocycles suitable for both production of CEST contrast and incorporation into liposomes and polymeric drug delivery systems. These CEST contrast agents can be synthesized, incorporated into the drug delivery systems to form the particles and screened for in vitro using MRI. More particularly, the CEST agents selected for the library are incorporated into particles containing a drug delivery system and a therapeutic agent, for example, stealth doxorubicin-loaded liposomes and stealth poly(lactic-co-glycolic acid)-co-polyethylene glycol (PLGA-PEG) drug delivery nanoparticles containing a cancer treatment drug such as paclitaxel.

The polymer-based PLGA-PEG particles can be used with respect to the example of treatment of cervical cancer, as these particles are able to rapidly penetrate human mucus secretions, allowing them to avoid rapid clearance from the vagina. Preventing rapid clearance from the vagina leads to greatly enhanced efficacy against cervical tumors as compared to conventional nanoparticles. MRI is then used to track the CEST labels such that the distribution and persistence of nanoparticles at the treatment site, such as, in the case of cervical cancer the cervicovaginal tract, can be monitored. It is also possible that “Multicolor” CEST imaging can be used to distinguish between two nanocarriers at once, with one carrier administered systemically, and a second administered locally.

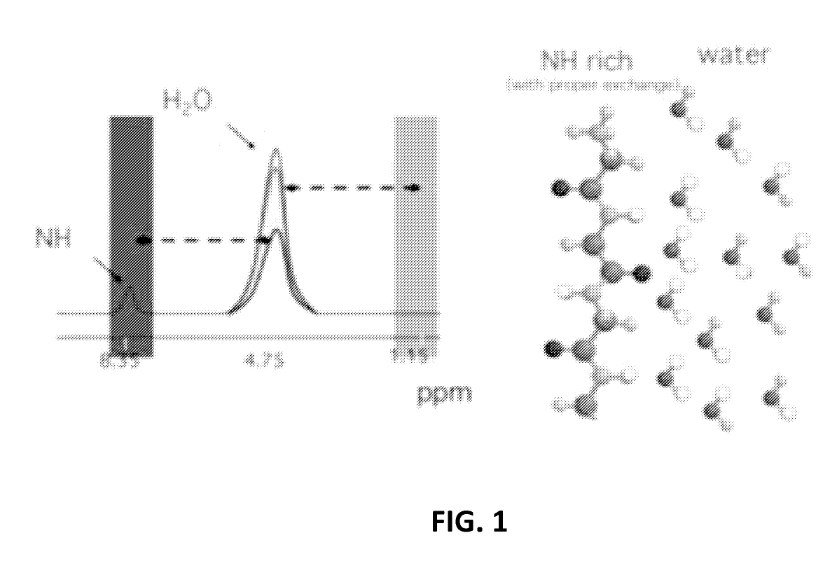

For the library of CEST agents, FIG. 1 illustrates the approach for detecting backbone amide NH protons in a peptide. In addition to these amide NH protons, a number of other exchangeable protons in proteins can be tuned to appropriate exchange rates for CEST contrast. For instance, the library of suitable peptides can include, but is not limited to, NH, guanidyl NH₂ and OH protons, and sugars with CEST-detectable OH groups (glycoCEST). Peptides and sugars are natural, bioorganic, biodegradable compounds.

Preferably, the library of CEST agents, according to the present invention, takes the form of a compilation of bioorganic agents that can produce CEST contrast in liposomal and polymeric nanoparticle delivery systems. The CEST agents are also, preferably, biocompatible. One proposed strategy is to encapsulate a DIACEST agent in the interior, saturate the exchangeable protons on the agents and allow transfer to water in the interior and, subsequently, the exterior of the particles. This two-hop exchange transfer process is expected to cause a fractional reduction of bulk water signal. It is important to note that, even though the chemical shift difference for the bioorganic agents is only a few ppm, this is often larger than the average shift of the water molecules when using a paraCEST agent. Another approach is to attach the bioorganic CEST agents around the periphery of the particles, where there will be more exposure to water.

Both cationic and neutral peptides that are rich in exchangeable protons with appropriate exchange rates (sufficiently slow to be magnetically labeled) can be utilized as CEST agents. Several natural proteins exhibit CEST properties that can be exploited. Additionally, species of the protamine family and also glycosaminoglycans have been imaged using CEST. This list will no doubt grow and should probably include multiple members of thehistone and cell penetrating peptide families, such as HIV-1 TAT. Macromolecules with multiple amide groups or imino groups can also be used to give sufficient CEST effect to allow the detection of agents in the micromolar range, which is sufficient for detection of contrast agents integrated into drug delivery particles. Multi-color imaging is expected to be useful for cell tracking (Project 3) as well as for monitoring tumor therapy, in particular for combination therapies such as the proposed combination of both systemic and local drug delivery to the cervical tumors (Aim 3B of this proposal).

The CEST agents are biocompatible/biodegradable and do not introduce foreign metals (or any metal) into the delivery particles. This has a high likelihood of reducing unwanted side effects from traditionally used metal based contrast agents. Use of suitable CEST imaging agents would also eliminate the need for radiation (e.g. 99 mTc-Doxil) for the two most commonly used types of drug delivery nanosystems, which is expected to greatly increase the use of particles capable of both imaging and therapy (“theragnostics”).

Preferably, the library is built around four different types of peptides: 1) ring NH-rich 2) backbone NH-rich, 3) guanidyl NH2-rich and 4) OH-rich because these protons have exchange rates which are potentially suitable for CEST contrast. Both empirical equations were used to estimate which amino acid sequences might produce CEST contrast as well as a high-throughput screening method (at a rate of about 20 compounds per NMR session using only 20 μL/agent for the measurements). A method called QUEST can also be used to measure the CEST agent exchange rates, which are subsequently used to fine tune the peptide sequences.

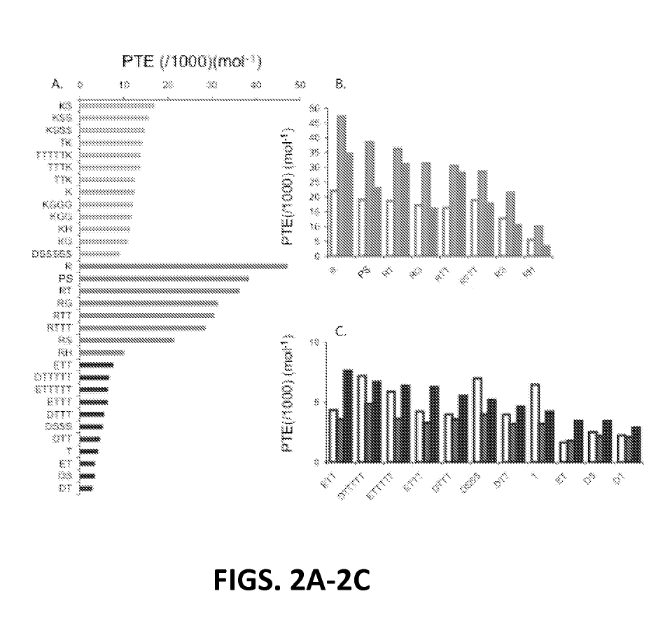

FIGS. 2A-2C, illustrate a list of 30 of such CEST peptides, and compares the sensitivity for three varieties: NH-rich (FIG. 2A), gNH2-rich (FIG. 2B), and OH-rich peptides (FIG. 2C). These agents include suitable amino acids from the library as well as lysine with suitable heterocycles linked to the sidechain (more detail below). One of the strengths of building the library around peptides is the ease of scaling up of the syntheses using peptide synthesizers. If the number of exchangeable protons per particle has to be increased due to inadequate MR contrast in vitro, the peptide length can be readily increased.

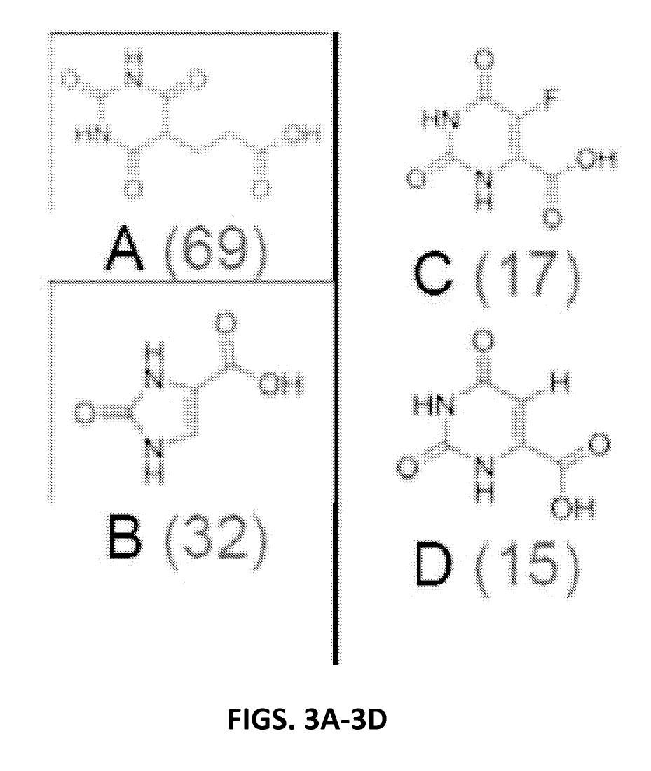

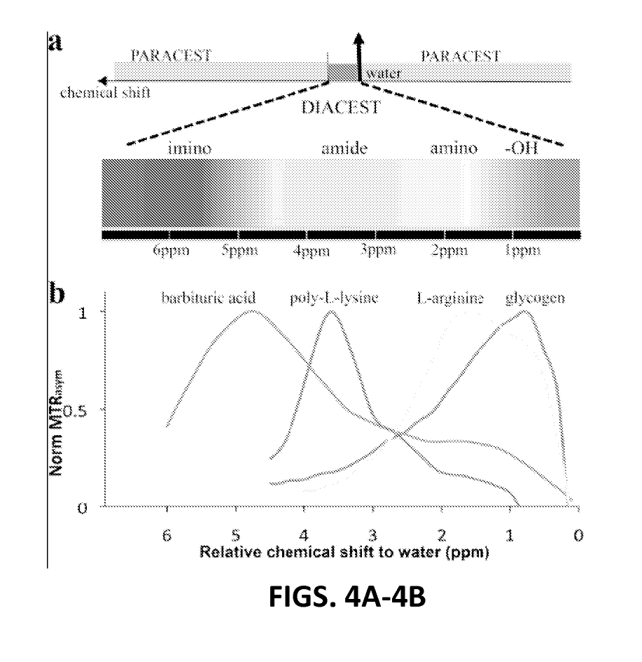

One disadvantage of the library is that the exchangeable protons are close in frequency to water (3.6 ppm or less). One way to improve CEST sensitivity in vivo is to increase the chemical shift in order to reduce the signal loss from direct saturation effects. Aromatic ring NH protons, such as those found in histidine, often resonate 4-5 ppm from water, or more, and as such would be good candidates. Efforts to obtain CEST contrast from histidine were unsuccessful until substitutions for ring protons were used. A series of modified pyrimidine and imidazole compounds were tested to determine the best substitutions. Four of these are shown in FIGS. 3A-3D. For comparison, the K peptide in FIG. 2A would produce ˜13% contrast for the same concentration of exchangeable protons. Substitutions at the 2-position in the imidazole ring result in large changes in CEST contrast, and also found 6 member rings that produce more contrast. As a result, the library of compounds will possess four different types of exchangeable protons to allow discrimination of the various nanocarriers using MRI. FIGS. 4A and 4B illustrates the CEST color spectrum for this range of exchangeable protons in vitro prior to incorporation into nanocarriers, demonstrating that the CEST contrast curves (MTRasym curves illustrated in FIG. 1) are quite different for these protons.

In order to apply similar reaction schemes for coupling both previous CEST peptides and these heterocycles to drug nanocarriers, the heterocycles will be linked to the lysine side chain (Scheme 1). This has been tested this on 2-oxo-2,3-dihydro-1H-imidazole-4-carboxylic acid (compound B in FIG. 2B). Reaction of compound B with N-hydroxysuccinimide (NHS) provided compound 1 in scheme 1 in 60% yield. Coupling of 1 with Lys (Boc)-COOH afforded compound 2 in 55% yield. The CEST contrast for compound 2 was ˜31%, comparable to compound B in the preliminary screening. It was also observed that a similar trend for a lysine analog substituted with orotic acid (compound D in Fig C2 and 3d in Scheme 1). Therefore, introduction of CEST moieties on the side chain of lysine will not significantly perturb the parent CEST contrast.

Fmoc-protected lysine analog (3a-d, Scheme 1) can be prepared utilizing the same strategy as compound 2. For instance, coupling of 1 with Lys (Fmoc)-COOH will give compound 3b. In addition, DOTA-attached (4) or fluorescein-attached (5) lysine analogs can be produced by this scheme. DOTA and fluorescein moieties will be used to control the hydrophilicity and measure the concentration of CEST agents when linked to nano-carriers. The preferred heterocycles are barbituric acid and imidazole, the top two in Table C.1.

TABLE C.1
Plan of DIACEST peptide syntheses (Years 1-4, Aim 1).

| Parent Compound | Why Selected | Types of modifications | |
|---|---|---|---|
| Barbituric Acid | Aromatic Ring NH | F ring | COOH ring |
| | Exchangeable proton | substitution | substitution |
| | | Carbonyl ring | phospholipid |
| | | substitution | attachment |
| | | Multiple copies | |
| | | in peptide | |
| Imidazole | Aromatic Ring NH | Br ring | COOH ring |
| | Exchangeable proton | substitution | substitution |
| | | Carbonyl ring | phospholipid |
| | | substitution | attachment |
| | | Multiple copies | |
| | | in peptide | |
| Lysine-Glycine | Backbone NH proton | Multiple copies | phospholipid |
| | | in peptide | attachment |
| L-Arginine | Guanidyl NH₂ proton | Multiple copies | phospholipid |
| | | in peptide | attachment |

In addition to the CEST agent library, the drug delivery system also must be considered. For example, with respect to cervical cancer, or any other disease where mucus must be penetrated, nanoparticle drug delivery systems densely coated with low molecular weight PEG are able to penetrate cervicovaginal mucus at rates as high as only 4-fold slower compared to their theoretical rates in water, whereas uncoated particles are ˜40,000-fold slower in mucus than in water. These “mucus penetrating particles” (MPP) were also shown to distribute more uniformally in the cervicovaginal tract and reach the vaginal folds (ruggae) within minutes after administration.

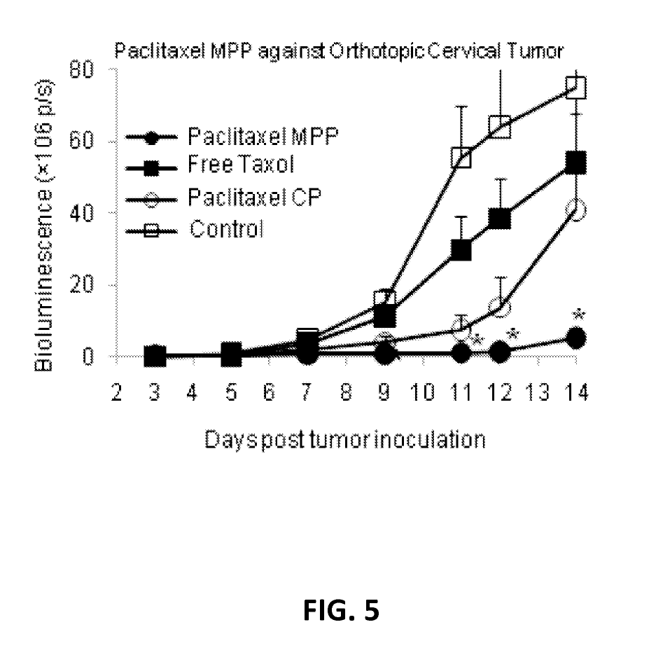

Biodegradable MPP can also be formed using materials composed entirely of FDA-approved monomers. These MPP are able to encapsulate and release low-molecular weight chemotherapeutics, including doxorubicin and paclitaxel, continuously for several days, thus providing local sustained delivery to the vaginal epithelium. Paclitaxel-loaded MPP inhibits tumor growth in an orthotopic murine cervical cancer model, while other local treatments, including paclitaxel-loaded CP and free Taxol®, are much less effective, as illustrated in FIG. 5. CEST imaging technology also allows for close monitoring of the administration and retention of particles near the tumors.

An alternative way to incorporate CEST agents into stealth liposomes is by covalently attaching them to the lipid membrane. Scheme 2 shows a general approach to conjugate CEST compounds to the 1,2-distearoyl-sn-glycero-3-phosphoethanolamine (DSPE) lipid (representative compound 7 is shown in Scheme 2). Commercially available Fmoc-Gly-preloaded Wang resin will be used as a starting material. Deprotection of Fmoc groups and coupling with carboxylic acid of lysine analogs (3a-3d, 4 and 5 in Scheme 1), in several SPPS cycles will afford resin-bound ligand peptides containing DIACEST moieties. Compound 6 in Scheme 2 is one of the examples. Coupling of 6 with DSPE-glutaric NHS ester, which will be prepared from DSPE and glutaric anhydride in 2 synthetic steps by applying the reported procedure. Subsequent removal of resin with trifluoroacetic acid and deprotection of NO2 group will give compound 7.95 Any of the DIACEST peptides can be coupled using this strategy, and will prepare labeled DSPE from the highest sensitivity peptides based on the CEST contrast screens. This will include using small peptides containing just 1 arginine and the glycine spacer or one of lysine analogs in scheme 1 plus glycine, to larger peptides which include DOTA (compound 4) or DOTA+ fluorescein (compound 4+5). Agents with anywhere from 1 to 12 or more residues long can be conjugated to DSPE, which will be important. If only small amounts of DSPE lipids can be incorporated into the lipid particles, the peptides can be elongated to increase the number of exchangeable protons/particle and improve the sensitivity.

PLGA-PEG will be activated using 4-Nitrophenyl chloroformate and subsequently conjugated to a tyrosine-containing crosslinker (4-[p-azidosalicylamido]butylamine) The presence of tyrosine at the terminal of polymers will allow 125I labeling after particles are formulated using standard iodination procedures.

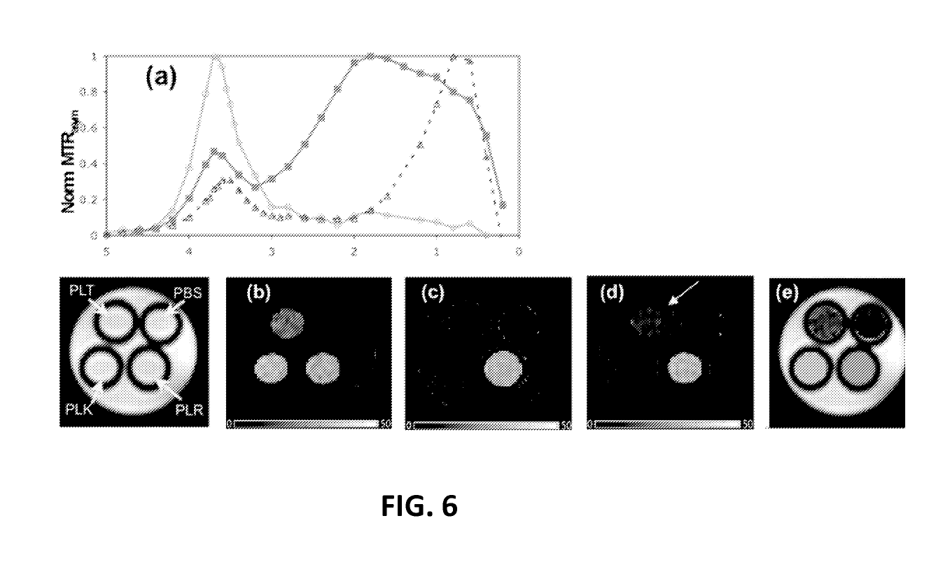

With respect to imaging and tracking of the drug delivery particles, paramagnetic MR contrast agents are generally limited to a single contrast (signal decrease or increase). However, peptide CEST agents can be designed with different types of exchangeable protons (i.e. with respect to NMR frequency) allowing them to be excited separately (in a similar fashion to fluorescent agents), termed “multicolor MR imaging”. FIG. 6 illustrates this using a phantom containing three different peptide-based CEST agents. This function-based design of polycationic peptides and neutral heterocycles, can be used for labeling nanoparticles and monitoring their location over time. Ultimately, each frequency could be used to label a different drug carrier, using the OH in serine or threonine, NH2 in arginine, asparagine or glutamine, the ring OH in tyrosine, and the ring NHs in tryptophan and histidine, for example.

### EXAMPLE

An exemplary implementation of the present invention is described herein, in order to further illustrate the present invention. The exemplary implementation is included merely as an example and is not meant to be considered limiting. Any implementation of the present invention on any suitable subject known to or conceivable by one of skill in the art could also be used, and is considered within the scope of this application.

A first exemplary particle includes a CEST agent incorporated into particles suitable for systemic chemotherapy. These will be incorporated into the interior and/or exterior (covalent conjugation) of liposomes and PLGA-PEG NPs. Two exemplary particles include but are not limited to: stealth liposomes encapsulating Larg, and stealth PLGA-PEG particles entrapping barbituric acid. CEST agents can also be incorporated into particles at different concentrations with the particles fully characterized for size, size distribution, surface charge, stability, and MRI in vitro contrast, and then promising formulations (with at least two levels of DIACEST agent concentration for each formulation) administered via the tail vein of C57BL/6 mice and imaged by MRI. The purpose of testing different concentrations of DIACEST agent is to optimize the concentration for obtaining sufficient contrast, meanwhile minimizing the possible influence of the contrast agent on drug loading and biodistribution of particles. The study design is summarized in Table C.2, below and optimization of dosing regimens is also summarized in Table C.3, below.

TABLE C.2
Summary of characterization of DIACEST-labeled particles
in vivo (Year 1 as example, Aims 2&3)*.

| Carrier | | Admin- | Animals/ | Animals/ | MRI Time |
|---|---|---|---|---|---|
| Type | Agent type | istration | group | year | points |
| Liposome | CEST | Systemic | 4 | 100 | 0, 6, 12, |
| or | compound | or Local | | | 24 and 36 |
| Polymeric | 1 (x2 | | | | hr post |
| | loading | | | | particle |
| | levels) | | | | admin- |
| | CEST | | 4 | | istration |
| | compound | | | | |
| | 2 (x2 | | | | |
| | leading | | | | |
| | levels) | | | | |
| | Unlabeled | | 4 | | |
| | SPECT + | | 5 | | |
| | MR | | | | |

TABLE C. 3
Optimize dosing regimens using CEST imaging
for improved efficacy (Year 5, Aim 3B)

| Systemic treatment | Local treatment | # of doses | Total # of animals |
|---|---|---|---|
| Selected systemic | Local placebo | 1, 2, or 3 | 24 (n = x per group) |
| CEST Chemo NP | NP | | |
| Systemic placebo | Selected local | 1, 2, or 3 | 24 (n = x per group) |
| NP | CEST Chemo NP | | |
| Selected systemic | Selected local | 1, 2, or 3 | 26 (n = x per group) |
| CEST Chemo NP | CEST Chemo NP | | |
| Systemic placebo | Local placebo | 1, 2, or 3 | 24 (n = x per group) |
| NP | NP | | |

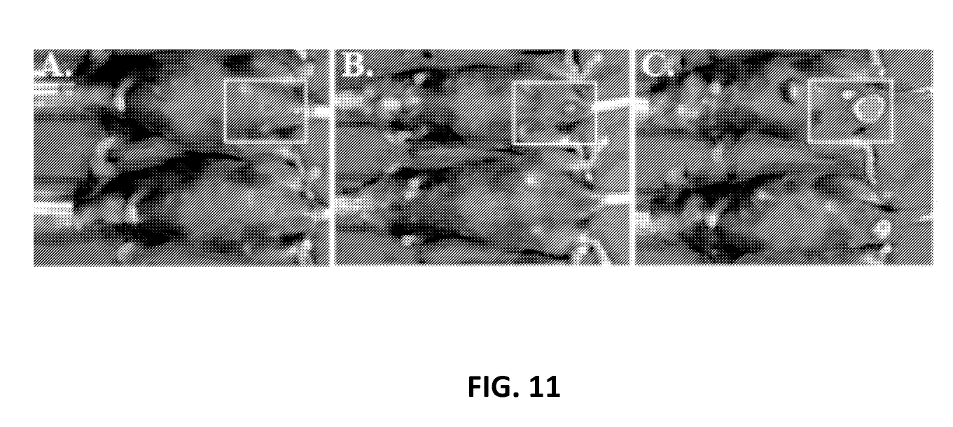

Unlabeled particles are used as negative control, and 125I and 111Inoxine labeled particles will be used for SPECT to validate MRI results. Tumor growth and treatment efficacy can be monitored by using in vivo bioluminescence imaging (BLI) and MRI similar to that shown in FIGS. 5, 11 & 12.

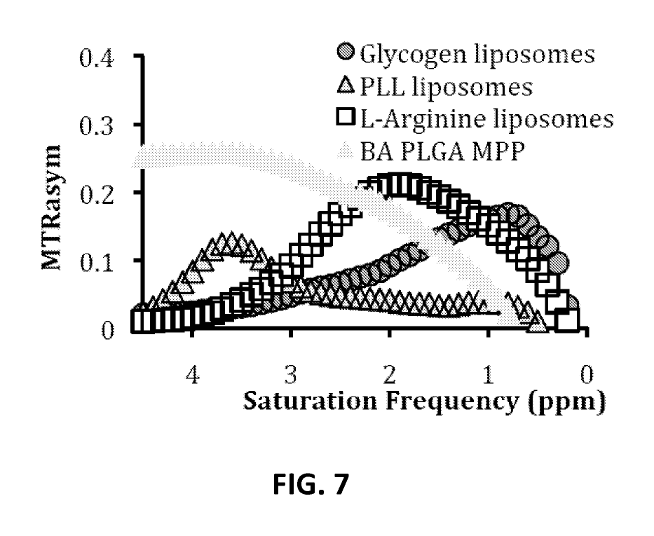

One way to prepare these particles is by inserting CEST agent in the interior of the particles. Results for three such DIACEST liposomes are illustrated in FIG. 7, prepared containing L-arginine (Larg), poly-L-lysine (PLL), and glycogen (Glyc), using starting solutions containing 100 mg/ml Larg, 25 mg/mL PLL (MW=15 kD) or 100 mg/ml Glyc (MW=25 k-100 k), respectively. The stealth liposomes were formed using the extended hydration method using Rhodamine attached to PE to fluorescently label these. They are then extruded through 50 nm cutoff filters and unencapsulated Larg, PLL or Glyc removed using dialysis with 250 kD cutoff PVDF dialysis tubing. The size and concentration of the liposomes were measured using dynamic light scattering and fluorescence with the final particle concentration ˜30 nM and the liposome size ˜100 nm. As can be seen from FIG. 7 these display frequency dependent CEST contrast similar to that of the unencapsulated agents, illustrated in FIGS. 4A and 4B.

Sensitivity reduces too rapidly due to CEST agent leakage from liposome Therefore, liposomes can also be prepared from the DIACEST labeled DSPE lipids described in section C.1.2 so that the nanocarrier is labeled only on the exterior leaving the interior for doxorubicin. The formulation can be optimized using either HSPC:Chol:DSPE-PEG2000:DSPE-CEST or POPC:Chol:DSPE-PEG2000:DSPE-CEST as component lipids. The liposomes will be formed using a modified extended hydration method. The concentration of DSPE-CEST can be varied from 10-40 mole % while adjusting the HSPC (or POPC) and Cholesterol ratio downward similar to that described previously for PARACEST liposomes. Previously described procedures can be slightly modified to encapsulate doxorubicin within these liposomes. Briefly, lipids will be hydrolyzed in NH4SO4 and doxorubicin will be loaded using a pH-gradient method 100. This strategy will produce liposomes with DIACEST agents covalently coupled to them removing the drop in particle sensitivity over time.

Two different types of PLGA-PEG based particles can also be prepared: those with DIACEST agent enclosed in the interior and a second type with CEST agent covalently attached to the periphery. They will be prepared using a nanoprecipitation method. For particles with DIACEST agents encapsulated in the interior, DIACEST agent (e.g. barbituric acid or other agents), polymer, and paclitaxel will be co-dissolved in acetonitrile and added dropwise into aqueous solution. Particles will then be collected by centrifugation after the organic solvent is removed and washed twice to remove unencapsulated agent and drug.

The in vitro sensitivity for incorporating barbituric acid into 200 nm stealth PLGA-PEG particles is illustrated in FIG. 7, compared to the DIACEST stealth liposomes prepared previously. As can be seen, the particles display CEST contrast (MTRasym) with a maximum around 3.8 ppm, but with a different frequency dependence from the liposome-based agents.

Sensitivity reduces too much over time due to agent leakage. Thereofore, PLGA-PEG particles can also be prepared with CEST agents covalently attached to the surface. Paclitaxel-encapsulated particles will be first formulated as described above, and the DIACEST peptides will be attached directly to the particles. These peptides will contain a N-terminal amine, which will react with the free carboxylic acid groups on the particles in the presence of EDC and NHS in PBS to generate the CEST-particle conjugates similar to schemes 1 & 2. The length of the peptides that are conjugated can be varied in order to tailor the CEST contrast of the particles, but without need for DOTA (used to increase solubility of lipid headgroup).

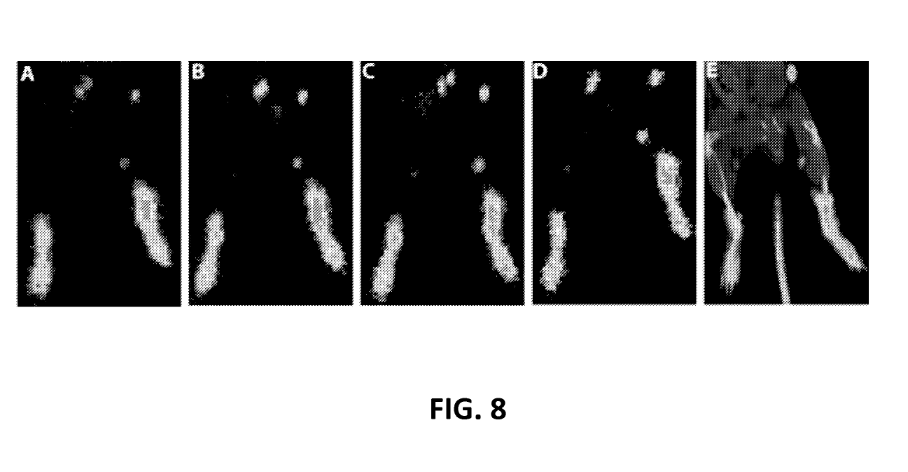

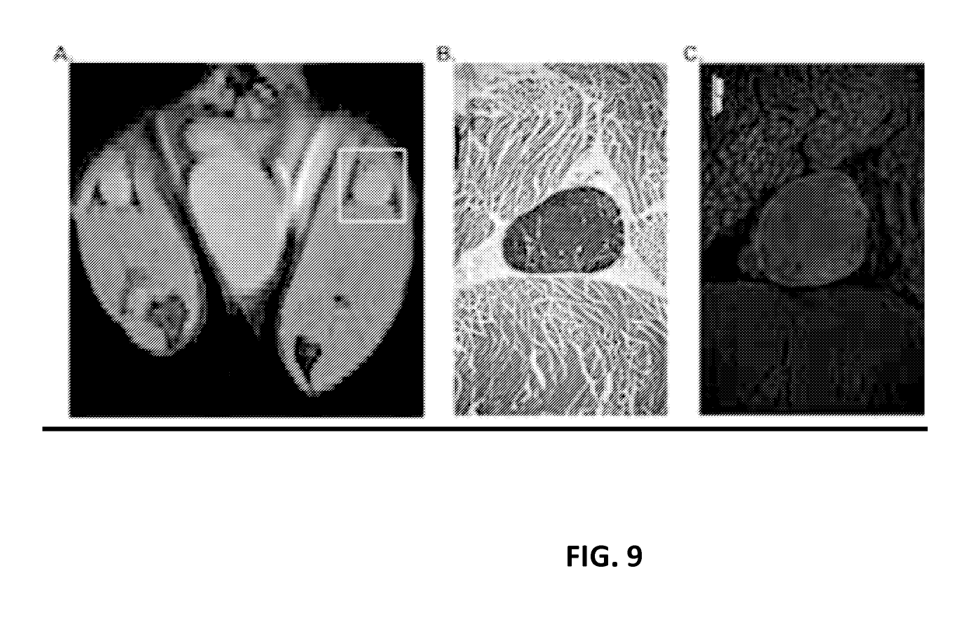

As a proof of principle, the passive lymphatic draining of liposomes was used to determine if the CEST particles could be detected in vivo and also to investigate the frequency dependence of the in vivo CEST contrast. NSPECT/CT was used to obtain preliminary images of DIACEST liposomes after subcutaneous injection into the footpad of mice. C57BL/6 mice were injected with 30 ul solutions containing 30 nM radioisotope labeled liposomes (containing ₁₁₁In oxine in the interior) with two different sizes (99 nm, 134 nm) injected (FIG. 8). It was determined that uptake was about 0.6% for the 99 nm liposomes in the popliteal lymph node 24 hrs post-injection. In addition, histology was performed to determine how the liposomes were distributed within these nodes (FIG. 9). As can be seen, the liposomes are largely within the ducts for this node but are also dispersed throughout.

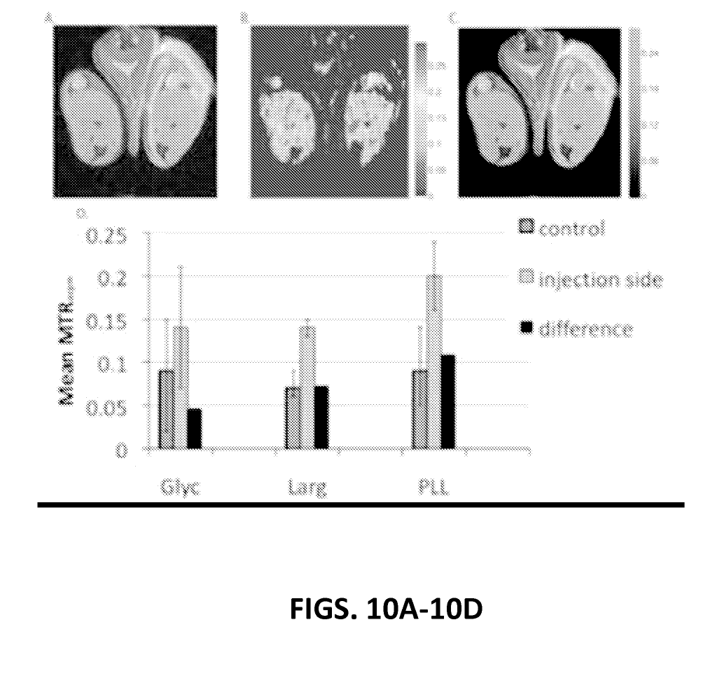

In vivo detection of endogenously expressed protein MR CEST agents in a mouse brain at 11.7 T can be tested using 9 L tumor cells and endogenous agents, such as cellular proteins/peptides and glycogen. The WASSR methodology for frequency referencing with respect to water can also be used improve these already acceptable data and allow detection of smaller concentrations. FIGS. 10A-10C, illustrate recently acquired in vivo liposome data. As shown by the SPECT data in FIG. 8, most of the liposomes remain near the injection site during the time period of the study, with the popliteal lymph node showing the highest uptake outside the foot.

The mice were injected in one foot with one type of DIACEST liposome (Glyc, Larg or PLL) each time, which allows the direct comparison with intact lymph nodes on the other side as control. The CEST imaging consists of three parts, image collection, WASSR correction, and CNR filtering and contour leveling. An example of the types of images acquired is illustrated in FIGS. 10A-10C for the PLL liposomes, which are clearly detected in the popliteal lymph node on the inject side. FIG. 10D illustrates the CEST contrast on the injection and control side for all three CEST formulations. Larg liposomes were chosen for the first in vivo studies based on these measurements and also on the expected improved safety of Larg (a nutritional supplement) over PLL. The concentration of Larg in this liposome formulation will be used as an initial concentration for the systemic studies, but other concentrations can also be tested.

C57BL/6 mice will be treated with Depo Provera at 15 mg/kg to induce thinning of the vaginal epithelium. Seven days post treatment, the vaginal epithelium will be disrupted using cytobrush and TC-1 tumor cells will be inoculated by intravaginal instillation. The growth of tumor cells will be monitored every two days by live animal bioluminescence imaging as shown in FIG. 11.

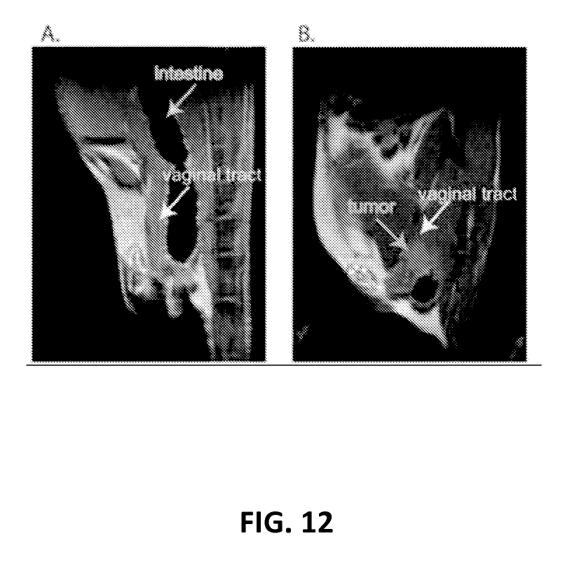

Determining the image contrast for TC-1 cervical tumors is key such that the tumor therapy can be monitored directly using MRI. Tumors are readily detectable on a 9.4 T Bruker scanner using T2-weighted imaging. For example, in FIG. 12, the tumor is localized high up in the vaginal tract. These tumors can also extend down to the entrance to this tract, lining the entire tract. An alternative technique to detect these tumors would be Amide Proton Transfer Imaging. This endogenous contrast will be separated from the exogenous agents by exchange filtering.

Particles are injected into the tail vein of tumor bearing mice and imaged using the methods described above. A series of studies can be executed using both PLGA-PEG particles and stealth liposomes. After the optimal loading of CEST agent is determined, quantitative CEST/SPECT images for 5 time points can be collected, including immediately post-injection, 6 hrs, 12 hrs, 1 day, and 36 hrs afterwards. The CEST contrast will be correlated with the SPECT for validating the nanoparticle retention and also correlated with tumor size (as determined by both MR and BLI) to determine if the CEST contrast can be used to predict therapeutic outcome. For validation, after the in vivo MRI/SPECT studies, the tumors can be excised to determine the amount of radiation in the tumors using a gamma counter. The blood-half life of the particles will be determined using 111In liquid scintillation counting (LSC) by taking blood plasma samples (tail vein), and excising the major organs (liver, spleen, kidney, lungs, intestines).

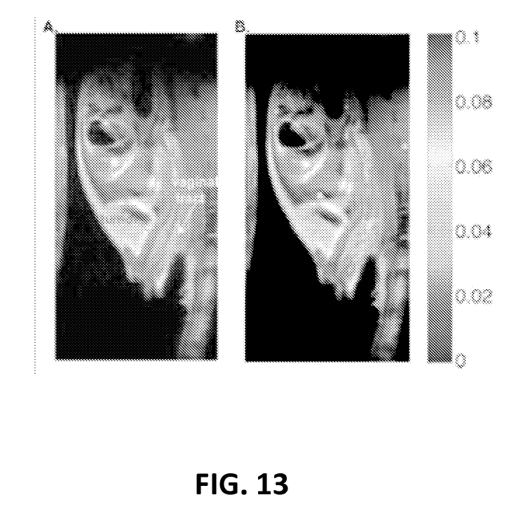

The particles can also be delivered intravaginally. FIG. 12 illustrates images of such a method, and FIG. 13 illustrates images of DIACEST PLGA-PEG particles administered locally. For local administration of particles in the cervicovaginal tract, tumor-bearing mice are first anesthetized and 10 uL of liposome or polymeric particle suspension will be instilled intravaginally using a pipette. FIG. 13 displays an in vivo CEST contrast image 12 hrs after administration. The CEST contrast is clearly detected proximal to the cervical tumor. A series of studies using both stealth liposomes and stealth PLGA particles can be done, with the number of animals, administration scheme and number of MRI/SPECT images summarized in Table C2. The first experiments are to determine how much CEST agent is necessary to load into the particles for in vivo MR detection. After this has been determined, 5 time points will be acquired for MRI/SPECT to determine how long the drug particles persist near the tumor as measured by both MR and SPECT and determine if the CEST contrast can be correlated with therapeutic outcome. For validation, the tumors can be excised for histology.

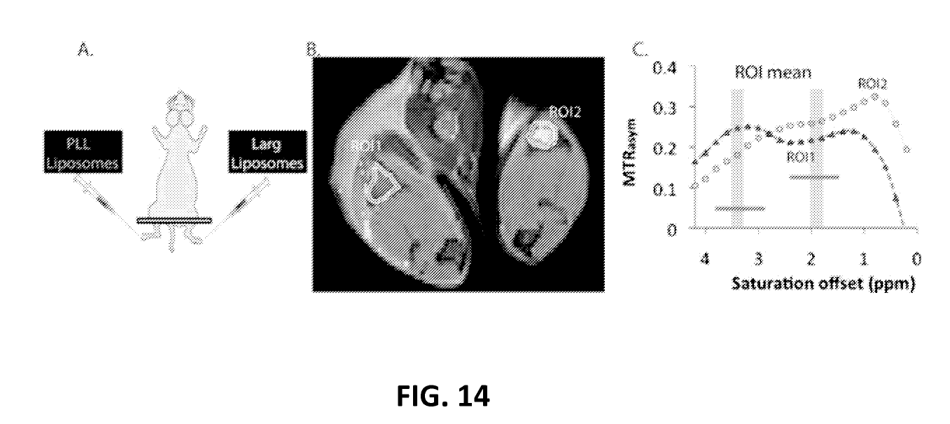

Simultaneous multi-color mapping of particles delivered via two different routes (systemic and local), can also be done using CEST imaging. This information on particle persistence can provide guidance on optimizing multiple dosing regimens that improve drug efficacy (Table C.3). FIG. 14 illustrates “multicolor” CEST imaging to discriminate between PLL and Larg liposomes. The amount of Paclitaxel and doxorubicin loaded particles in the vicinity of the tumor independently in vivo can be determined using this information.

Selected DIACEST particles containing chemo drugs can also be tested with the DIACEST label attached to either 1) systemic administered particles only, 2) local administered particles only, or 3) both systemic+ local particles labeled. Multiple (up to 3) doses will be given to improve the overall efficacy and the frequency of dosage will be optimized.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

1. An agent for use in conjunction with magnetic resonance (MR) imaging comprising:
a biocompatible chemical exchange saturation transfer (CEST) agent, said biocompatible CEST agent being configured to create contrast in an MR image, wherein the contrast is detectable using a saturation transfer CEST method of MR imaging;
a drug delivery system;
a therapeutic agent; and
wherein the biodegradable CEST agent, the drug delivery system, and therapeutic agent are combined to form a particle.

2. The agent of claim 1 wherein the particle further comprises a nanoparticle.

3. The agent of claim 1 wherein the CEST agent comprises a non-metallic DIACEST agent.

4. The agent of claim 1 wherein the agent is configured for use as systemic nanoparticle-based chemotherapy.

5. The agent of claim 1 wherein the agent is configured for use as local nanoparticle-based chemotherapy.

6. The agent of claim 1 wherein the CEST agent further comprises at least one of a polypeptide or organic heterocycle.

7. The agent of claim 1 wherein the CEST agent further comprises at least one of a peptide that is ring NH-rich, backbone NH-rich, guanidyl NH₂-rich, or OH-rich.

8. The agent of claim 1 wherein the CEST agent further comprises a macromolecule with multiple amide or imino groups.

9. The agent of claim 1 wherein the drug delivery system comprises a mucus penetrating particle.

10. The agent of claim 1 wherein the drug delivery system comprises one of a stealth liposome or a stealth poly(lactic-co-glycolic acid)-co-polyethylene glycol (PLGA-PEG) particle.

11. The agent of claim 1 wherein the therapeutic agent takes the form of one of doxorubicin or paclitaxel.

12. A method for tracking a delivery of a therapeutic agent in a subject comprising:
providing a particle containing a biocompatible CEST contrast agent, a therapeutic agent, and a drug delivery system, wherein the biocompatible CEST agent is configured to create contrast in an MR image detectable using a saturation transfer CEST method of MR imaging;
delivering the particle to the subject;
using the saturation transfer CEST method of MR imaging to obtain an MR image of the subject; and
using the MR image of the subject to track the delivery of the therapeutic agent using the contrast created by the CEST agent.

13. The method of claim 12 further comprising using the particle to treat cancer.

14. The method of claim 13 further comprising using the particle to treat cervical cancer.

15. The method of claim 12 further comprising delivering the particle to the subject systemically.

16. The method of claim 12 further comprising delivering the particle to the subject locally.

17. The method of claim 12 further comprising building a library of available CEST contrast agents for use in conjunction with the method.

18. The method of claim 12 further comprising using multicolor MR imaging.

19. The method of claim 12 further comprising using information from the image of the subject to determine dose frequency for the therapeutic agent.

20. The method of claim 12 further comprising using information from the image of the subject to determine clearance of the therapeutic agent.
